(19) United States
(12) Patent Application Publication
(10) Pub. No.: US 2023/0230681 A1
(43) Pub. Date: Jul. 20, 2023

(54) METHODS, SYSTEMS, AND COMPUTER PROGRAM PRODUCTS FOR GENERATING A PROVIDER REFERRAL RECOMMENDATION BASED ON A MULTI-FACTOR REFERRAL SUCCESS METRIC

(71) Applicant: Change Healthcare Holdings LLC, Nashville, TN (US)

(72) Inventor: Jonathan Watters, Abington, MA (US)

(21) Appl. No.: 17/648,249

(22) Filed: Jan. 18, 2022

(51) Int. Cl. G16H 40/20; G16H 50/20; G06Q 30/02

(52) U.S. Cl. CPC G16H 40/20; G16H 50/20; G06Q 30/0283

(57) ABSTRACT

A method includes receiving first information associated with a patient, the first information including a procedure or treatment that is recommended for the patient; and generating a list of provider candidates for referring to the patient based on the first information associated with the patient, the list of provider candidates being ranked based on a multi-factor referral success metric. The multi-factor referral success metric comprises a cost factor, a complication factor, and a quality factor.

### FIELD

The present inventive concepts relate generally to health care systems and services and, more particularly, to the use of support systems that can be used by health care providers for referring patients to other providers.

### BACKGROUND

In providing health care services to patients, health care service providers frequently have the need to refer patients to other providers for certain treatments or evaluations. Health care service providers may be provided with a provider directory, which may be organized by specialty, but may be provided with little additional assistance in selecting a particular provider to which to refer a patient. As a result, health care service providers may rely primarily on anecdotal information, such as familiarity, in selecting a provider to which to refer a patient or use lists that may be provided by payors identifying in network candidates, which can lower costs for patients.

### SUMMARY

According to some embodiments of the inventive concept, a method comprises: receiving first information associated with a patient, the first information including a procedure or treatment that is recommended for the patient; and generating a list of provider candidates for referring to the patient based on the first information associated with the patient, the list of provider candidates being ranked based on a multi-factor referral success metric. The multi-factor referral success metric comprises a cost factor, a complication factor, and a quality factor.

In other embodiments, the first information further includes geographic information associated with the patient.

In still other embodiments, the complication factor comprises: unanticipated problems that have arisen following, and are a result of, one or more previous procedures, treatments, or patient illnesses.

In still other embodiments, the cost factor comprises: expenses associated with past performance of the procedure or the treatment that is recommended for the patient.

In still other embodiments, the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

In still other embodiments, generating the list of provider candidates comprises generating the list of provider candidates using an artificial intelligence engine.

In still other embodiments, the method further comprises identifying, for each of the provider candidates, one factor of the multi-factor referral success metric that most contributes to the respective provider candidate's rank in the list of provider candidates.

In still other embodiments, the method further comprises compiling first cost of care information for a first plurality of patients that have received referrals to one or more first providers that were selected based on a plurality of lists of provider candidates generated therefor and ranked based on the multi-factor referral success metric, respectively; and communicating the first cost of care information to one or more payors for the first plurality of patients.

In still other embodiments, the method further comprises compiling second cost of care information for a second plurality of patients that have received referrals to one or more second providers that were not selected based on a plurality of lists of provider candidates generated therefor and ranked based on the multi-factor referral success metric, respectively; comparing the second cost of care information with the first cost of care information to determine a trend in the difference between the second cost of care information and the first cost of care information over a time period; and communicating the trend to the one or more payors for the first plurality of patients or one or more payors for the second plurality of patients.

In some embodiments of the inventive concept, a system comprises a processor; and a memory coupled to the processor and comprising computer readable program code embodied in the memory that is executable by the processor to perform operations comprising: receiving first information associated with a patient, the first information including a procedure or treatment that is recommended for the patient; and generating a list of provider candidates for referring to the patient based on the first information associated with the patient, the list of provider candidates being ranked based on a multi-factor referral success metric. The multi-factor referral success metric comprises a cost factor, a complication factor, and a quality factor.

In further embodiments, the first information further includes geographic information associated with the patient; the complication factor comprises: unanticipated problems that have arisen following, and are a result of, one or more previous procedures, treatments, or patient illnesses; the cost factor comprises: expenses associated with past performance of the procedure or the treatment that is recommended for the patient; and the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

In still further embodiments, generating the list of provider candidates comprises generating the list of provider candidates using an artificial intelligence engine.

In still further embodiments, the operations further comprise: identifying, for each of the provider candidates, one factor of the multi-factor referral success metric that most contributes to the respective provider candidate's rank in the list of provider candidates.

In still further embodiments, the operations further comprise: compiling first cost of care information for a first plurality of patients that have received referrals to one or more first providers that were selected based on a plurality of lists of provider candidates generated therefor and ranked based on the multi-factor referral success metric, respectively; and communicating the first cost of care information to one or more payors for the first plurality of patients.

In still further embodiments, the operations further comprise: compiling second cost of care information for a second plurality of patients that have received referrals to one or more second providers that were not selected based on a plurality of lists of provider candidates generated therefor and ranked based on the multi-factor referral success metric, respectively; comparing the second cost of care information with the first cost of care information to determine a trend in the difference between the second cost of care information and the first cost of care information over a time period; and communicating the trend to the one or more payors for the first plurality of patients or one or more payors for the second plurality of patients.

In some embodiments of the inventive concept, a computer program product comprises a non-transitory computer readable storage medium comprising computer readable program code embodied in the medium that is executable by a processor to perform operations comprising: receiving first information associated with a patient, the first information including a procedure or treatment that is recommended for the patient; and generating a list of provider candidates for referring to the patient based on the first information associated with the patient, the list of provider candidates being ranked based on a multi-factor referral success metric. The multi-factor referral success metric comprises a cost factor, a complication factor, and a quality factor.

In other embodiments, the first information further includes geographic information associated with the patient; the complication factor comprises: unanticipated problems that have arisen following, and are a result of, one or more previous procedures, treatments, or patient illnesses; the cost factor comprises: expenses associated with past performance of the procedure or the treatment that is recommended for the patient; and the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

In still other embodiments, generating the list of provider candidates comprises generating the list of provider candidates using an artificial intelligence engine.

In still other embodiments, the operations further comprise: identifying, for each of the provider candidates, one factor of the multi-factor referral success metric that most contributes to the respective provider candidate's rank in the list of provider candidates.

In still other embodiments, the operations further comprise: compiling first cost of care information for a first plurality of patients that have received referrals to one or more first providers that were selected based on a plurality of lists of provider candidates generated therefor and ranked based on the multi-factor referral success metric, respectively; compiling second cost of care information for a second plurality of patients that have received referrals to one or more second providers that were not selected based on a plurality of lists of provider candidates generated therefor and ranked based on the multi-factor referral success metric, respectively; comparing the second cost of care information with the first cost of care information to determine a trend in the difference between the second cost of care information and the first cost of care information over a time period; and communicating the first cost of care information, the second cost of care information, and the trend to one or more payors for the first plurality of patients or one or more payors for the second plurality of patients.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a provider referral recommendation system based on a multi-factor referral success metric in accordance with some embodiments of the inventive concept;

FIG. 2 is a block diagram of an Artificial Intelligence (AI) implementation of the provider referral recommendation system of FIG. 1 in accordance with some embodiments of the inventive concept;

FIGS. 3-7 are flowcharts that illustrate operations for generating a patient referral recommendation using the provider referral recommendation system of FIG. 1 in accordance with some embodiments of the inventive concept;

FIG. 8 is a data processing system that may be used to implement one or more servers in the provider referral recommendation system of FIG. 1 in accordance with some embodiments of the inventive concept; and

FIG. 9 is a block diagram that illustrates a software/hardware architecture for use in the provider referral recommendation system of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components, and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

Embodiments of the inventive concept are described herein in the context of a referral recommendation engine for referring patients to providers in a health care environment that includes a machine learning engine and an artificial intelligence (AI) engine. It will be understood that embodiments of the inventive concept are not limited to a machine learning implementation of the referral recommendation engine and other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons. In other embodiments, the referral recommendation engine may be implemented without using AI using procedural and/or objected oriented computer readable program code, for example, in combination with processing and networking elements.

Health care services, e.g., treatments, examinations, diagnostics, prescriptions, and the like, are typically delivered by providers under two different types of models. One model is generally known as a fee-for-service model. In this case, a provider invoices a payor for each service rendered, which has led to concerns that the arrangement tends to incentivize providers to perform more services resulting in higher health care costs. Another model is known as value-based care (VBC). Under the VBC model, providers enter may into agreements with payors for patient care, in which the provider is paid a predetermined amount periodically, e.g., monthly or quarterly. When the cost of care exceeds the predetermined fee arrangement, the provider and payor absorb the loss according to a pre-arranged risk allocation. Similarly, when the cost of care falls below the predetermined fee arrangement, the provider and payor share the profit according to a pre-arranged profit-sharing agreement. Proponents of the VBC model often maintain that it rewards better health care outcomes (i.e., better quality care with fewer complications at lower costs) as opposed to rewarding the number of services provided.

Some embodiments of the inventive concept stem from a realization that, in a health care delivery environment, selecting a provider to which to refer a patient may be a complex problem that may involve a combination of a variety of considerations including, for example, clinical judgment (which specialty should the referred provider practice in based on the patient's problem list, clinical observations, and existing diagnoses), professional judgment (which provider among candidate providers for a referral is the best at fulfilling the diagnostic, procedural, and/or treatment needs of the patient), administrative factors (which provider is in the patient's insurance network and/or is in the referring provider's practice or health system), and experience factors (which provider communicates well with the referring provider, provides a good patient experience, and/or is convenient to the patient). A carefully considered referral may be especially important in VBC delivery models as high-cost providers, providers that deliver poor quality, and/or providers that often have higher rates of complications associated with their performance of procedures may result in higher overall health care costs, which can result in losses for either the primary care provider and/or payor.

Embodiments of the inventive concept may provide a referral recommendation system that may allow a user, e.g., a primary care provider, to enter information, such as a procedure or treatment that is recommended for a patient, and may generate a list of provider candidates for referring to the patient for further care. The list of provider candidates may be ranked based on a multi-factor referral success metric. The multi-factor referral success metric may comprise a cost factor, a complication factor, and a quality factor. In some embodiments, the list of provider candidates may be constrained based on a particular geographic range. For example, the primary care provider may provide a zip code for the patient for whom the referral pertains, or, in other embodiments, an interface application may be integrated with the system that manages the patient's electronic medical records or may be capable of obtaining information currently on a screen of a primary care provider's computer or other device for managing the patient's medical chart and the patient's home address or zip code may be obtained from one of these sources.

In accordance with various embodiments of the inventive concept, the complication factor may comprise unanticipated problems that have arisen following, and are a result of, one or more previous procedures, treatments, or patient illnesses and the cost factor may comprise expenses associated with past performance of the procedure or the treatment that is recommended for the patient. The quality factor may comprise a plurality of quality sub-factors including, but not limited to, an effectiveness of care sub-factor, an availability of care sub-factor, an experience of care sub-factor, a utilization sub-factor, a resource use sub-factor, and a health plan information sub-factor. To assist the user, e.g., primary care provider, in selecting a provider candidate for a referral, the factor that most contributes to a provider candidate's rank may be identified for each of the ranked provider candidates. The information or data for the various factors in the multi-factor referral success metric may be obtained from a variety of sources including, but not limited to, payors, patient medical records, patient feedback sources, provider feedback sources, professional certification bodies, and the like.

In some embodiments, the list of ranked provider candidates may be generated through use of a referral recommendation engine that is based on procedural and/or object-oriented computer readable program code in combination with one or more processing and/or networking elements. In some embodiments, the referral recommendation engine may be implemented or assisted through an AI engine.

Payors, for example, may seek information on which providers use the referral recommendation system according to embodiments of the inventive concept to evaluate whether the cost of care is decreasing for those patients who receive provider referrals that were identified through the provider referral recommendation system. Thus, cost of care information may be compiled for patients that have received referrals that were selected from a ranked list generated by the provider referral recommendation system according to embodiments of the inventive concept and cost of care information may be compiled for patients that have received referrals that were selected without using the provider referral recommendation system according to embodiments of the inventive concept. A trend may be determined based on the difference between the cost of care of patients that received provider referrals using the provider referral recommendation system and those patients that received provider referrals without using the provider referral recommendation system over time. The trend, and the cost of care information for the patients that received provider referrals through the provider referral recommendation system, and/or the cost of care information for the patients that received provider referrals without using the provider referral recommendation system may be communicated to one or more payors for the patients that received provider referrals using the provider referral recommendation system and/or for one or more payors for patients that received provider referrals without using the provider referral recommendation system. Payors may then evaluate the effectiveness of the provider referral recommendation system and, based on this evaluation, they may encourage primary care providers to adopt the system, particularly when they are engaged in a VBC relationship.

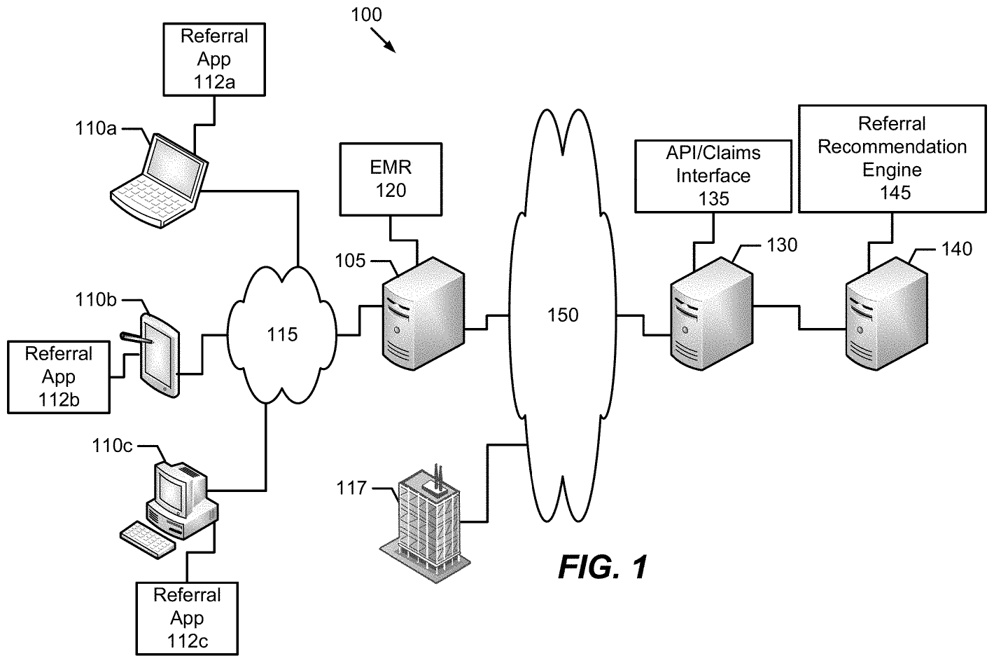

Referring to FIG. 1, a communication network 100 including a provider referral recommendation system, in accordance with some embodiments of the inventive concept, comprises a health care facility server 105 that is coupled to devices 110a, 110b, and 110c via a network 115. The health care facility may be any type of health care or medical facility, such as a hospital, doctor's office, specialty center (e.g., surgical center, orthopedic center, laboratory center etc.), or the like. The health care facility server 105 may be configured with an Electronic Medical Record (EMR) system module 120 to manage patient files and facilitate the entry of orders for patients via health care service providers (“providers”). Although shown as one combined system in FIG. 1, it will be understood that some health care facilities use separate systems for electronic medical record management and order entry management. The providers may use devices, such as devices 110a, 110b, and 110c to manage patients' electronic records and to issue orders for the patients through the EMR system 120. The devices 110a, 110b, and 110c may include referral applications 112a, 112b, and 112c that execute thereon and provide an interface for health care professionals to use the provider referral recommendation system. An order may include, but is not limited to, a treatment, a procedure (e.g., surgical procedure, physical therapy procedure, radiologic/imaging procedure, etc.) a test, a prescription, and the like. The network 115 communicatively couples the devices 110a, 110b, and 110c to the health care facility server 105. The network 115 may comprise one or more local or wireless networks to communicate with the health care facility server 105 when the health care facility server 105 is located in or proximate to the health care facility. When the health care facility server 105 is in a remote location from the health care facility, such as part of a cloud computing system or at a central computing center, then the network 115 may include one or more wide area or global networks, such as the Internet.

The communication network may include one or more payors 117, which represent private or public entities, such as insurers, that provide payments to providers for health care services rendered to patients based on claims submitted by the providers.

The provider referral recommendation system may include a health care facility/payor interface server 130, which includes an API/Claims interface module 135 to facilitate the transfer of information between the devices 110a, 110b, and 110c by way of the health care facility server 105 and the EMR system module 120, which the providers use to manage patient care, manage patient records and issue orders, and a referral server 140, which includes a referral recommendation engine module 145. The referral server 140 and referral recommendation engine module 145 may be configured to receive, for example, a procedure name that is input by a health care professional, such as a primary care provider. The referral server 140 may also be configured to receive geographic information associated with a patient for whom the procedure is intended. This geographic information may be entered by a provider using the devices 110a, 110b, and 110c and/or the referral applications 112a, 112b, and 112c may be integrated with the EMR system module 120 to obtain the patient geographic information therefrom or may be configured to parse information contained on the screen of the devices 110a, 110b, and 110c to obtain the patient geographic information. The referral server 140 may also be configured to obtain claim information from the one or more payors 117 along with information or data from other sources, such as patient medical record data from the EMR system module 120, information from patient feedback sources, information from provider feedback sources, information from professional certification bodies, and the like and may use this information or data in evaluating a multi-factor referral success metric for ranking provider candidates for referring to the patient for the procedure. Accordingly, the API/Claims interface module 135 in conjunction with the referral recommendation engine module 145 may be further configured to generate a list of provider candidates to which to refer a patient, which are ranked based on a multi-factor referral success metric. It will be understood that the division of functionality described herein between the referral server 140/referral recommendation engine module 145 and the health care facility/payor interface server 130/API/Claims interface module 135 is an example. Various functionality and capabilities can be moved between the referral server 140/referral recommendation engine module 145 and the health care facility/payor interface server 130/API/Claims interface module 135 in accordance with different embodiments of the inventive concept. Moreover, in some embodiments, the referral server 140/referral recommendation engine module 145 and the health care facility/payor interface server 130/API/Claims interface module 135 may be merged as a single logical and/or physical entity.

A network 150 couples the health care facility server 105 and the one or more payors 117 to the health care facility/payor interface server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The service provided through the health care facility/payor interface server 130, API/Claims interface module 135, referral server 140, and referral recommendation engine module 145 to provide provider referral recommendations based on a multi-factor referral success metric may, in some embodiments, be embodied as a cloud service. For example, health care facilities may integrate their EMR systems/order systems with the provider referral recommendation service and access the service as a Web service. In some embodiments, the provider referral recommendation service may be implemented as a Representational State Transfer Web Service (RESTful Web service).

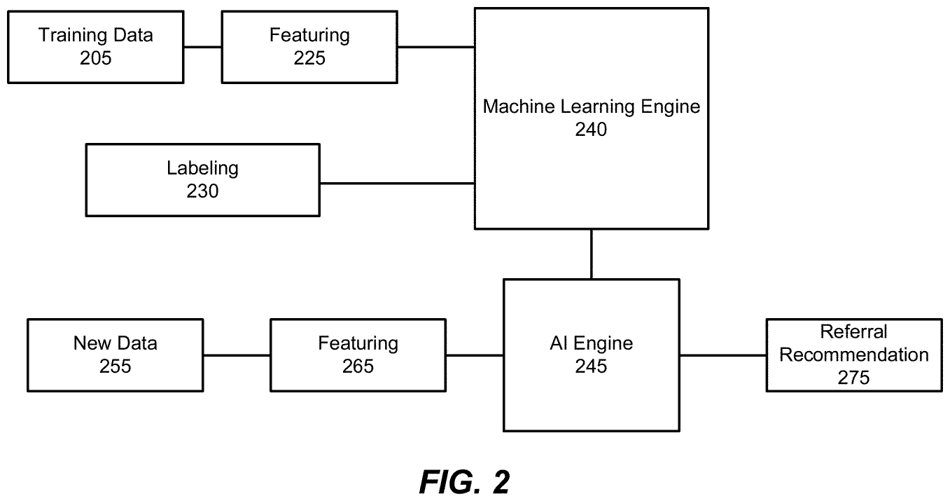

In some embodiments, the referral recommendation engine 145 may be configured to generate a list of ranked provider candidates through use of procedural and/or object-oriented computer readable program code in combination with one or more processing and/or networking elements. In other embodiments, the referral recommendation engine 145 may be implemented or assisted through an AI engine. FIG. 2 is a block diagram of the referral recommendation engine 145 used in an AI assisted provider referral recommendation system in accordance with some embodiments of the inventive concept. As shown in FIG. 2, the referral recommendation engine 145 may include both training modules and modules used for processing new data on which to make provider referral recommendations. The modules used in the training portion of the referral recommendation engine 145 include the training data 205, the featuring module 225, the labeling module 230, and the machine learning engine 240. The training data 205 may comprise information associated with both providers and patients and may relate to one or more factors in the multi-factor referral success metric used to rank providers for a referral. These factors may include a cost factor, a complication factor, and a quality factor. The cost factor may include economic information and may be based on claims data that have been issued by providers in the past for particular procedures or patient treatments. The complication factor may comprise information on unanticipated problems that have arisen following, and are a result of, one or more previous procedures, treatments, or patient illnesses. The quality factor may comprise information on a plurality of quality sub-factors including, but not limited to, an effectiveness of care sub-factor, an availability of care sub-factor, an experience of care sub-factor, a utilization sub-factor, a resource use sub-factor, and a health plan information sub-factor. The information or data for the various factors in the multi-factor referral success metric may be obtained from a variety of sources including, but not limited to, payors, patient medical records, patient feedback sources, provider feedback sources, professional certification bodies, and the like. The featuring module 225 is configured to identify the individual independent variables that are used by the referral recommendation engine 145 to make recommendations, e.g., through the generation of a ranked or prioritized list of providers, which may be considered a dependent variable. For example, the training data 205 may be generally unprocessed or formatted and include extra information in addition to cost factor information, complication factor information and quality factor information. For example, the medical claim data may include account codes, business address information, and the like, which can be filtered out by the featuring module 225. The features extracted from the training data 205 may be called attributes and the number of features may be called the dimension. The labeling module 230 may be configured to assign defined labels to the featured training data and to the generated recommendations to ensure a consistent naming convention for both the input features and the output recommendations, which may include both a list of ranked or prioritized provider candidates for a referral. The machine learning engine 240 may process the featured training data 205, including the labels provided by the labeling module 230, and may be configured to test numerous functions to establish a quantitative relationship between the featured and labeled input data and the referral recommendation outputs. The machine learning engine 240 may use regression techniques to evaluate the effects of various input data features on the referral recommendation outputs where the referral recommendation outputs are designed to improve or maximize a multi-factor referral success metric. These effects may then be used to tune and refine the quantitative relationship between the featured and labeled input data and the referral recommendation outputs. The tuned and refined quantitative relationship between the featured and labeled input data generated by the machine learning engine 240 is output for use in the AI engine 245. The machine learning engine 240 may be referred to as a machine learning algorithm.

The modules used for processing new data on which to make referral recommendations/outreach program recommendations include the new data 255, the featuring module 265, the AI engine module 245, and the referral recommendation module 275. The new data 255 may be at least a portion of the data/information used as the training data 205 in content and form except the data will be used for an actual referral recommendation and/or outreach program recommendation. For example, the new data 255 may include a procedure description and geographic information on the patient for whom a referral recommendation is being generated. Likewise, the featuring module 265 performs the same functionality on the new data 255 as the featuring module 225 performs on the training data 205. The AI engine 245 may, in effect, be generated by the machine learning engine 240 in the form of the quantitative relationship determined between the featured and labeled input data and the referral recommendation outputs. The AI engine 245 may, in some embodiments, be referred to as an AI model. The AI engine 245 may be configured to output referral recommendations in the form of a ranked list of provider candidates via the referral recommendation module 275. The referral recommendation module 275 may be configured to communicate the referral recommendation outputs in a variety of display formats.

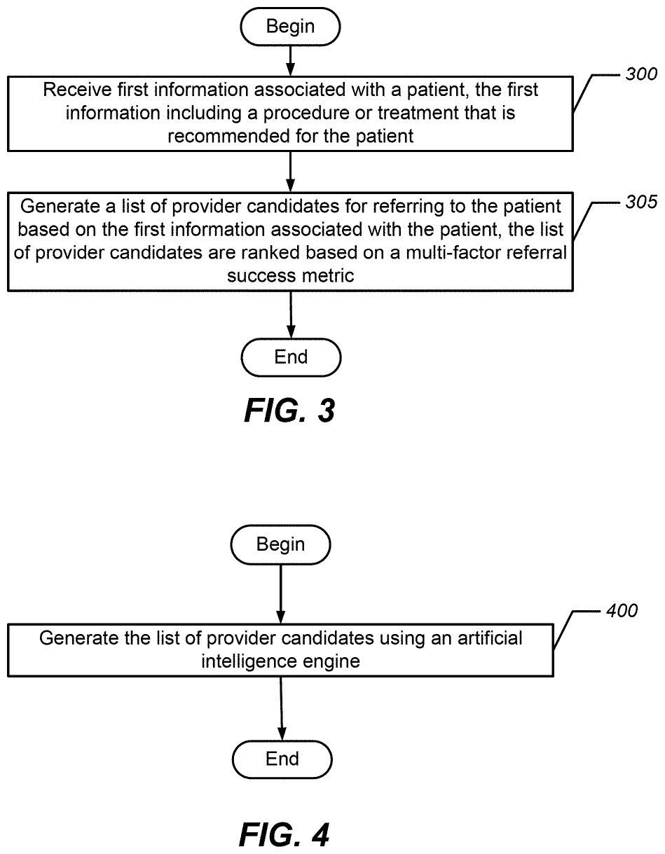

FIGS. 3-7 are flowcharts that illustrate operations for generating a patient referral recommendation using the provider referral recommendation system of FIG. 1 in accordance with some embodiments of the inventive concept. Referring now to FIG. 3, operations begin at block 300 where first information associated with a patient is received, which includes information regarding a procedure or treatment that is recommended for the patient. A list of provider candidates for referring to the patient is generated at block 305 based on the first information associated with the patient, which may include the procedure or treatment along with geographic information associated with the patient. The provider candidates in the list are ranked based on a multi-factor success metric. The multi-factor referral success metric may comprise a cost factor, a complication factor, and a quality factor.

In accordance with various embodiments of the inventive concept, the cost factor may comprise expenses associated with past performance of the procedure or the treatment that is recommended for the patient. The complication factor may comprise unanticipated problems that have arisen following, and are a result of, one or more previous procedures, treatments, or patient illnesses and. The quality factor may comprise a plurality of quality sub-factors including, but not limited to, an effectiveness of care sub-factor, an availability of care sub-factor, an experience of care sub-factor, a utilization sub-factor, a resource use sub-factor, and a health plan information sub-factor. The effectiveness of care sub-factor may include information related to patient care, such as, but not limited to, immunizations, cancer screenings, diabetes care, weight assessment and/or appropriate treatment for acute and chronic illnesses. The availability of care sub-factor may include information related to access to health care services including, but not limited to, adult access to preventive/ambulatory services, access to an annual dental visit, and children's access to a primary care provider. The experience of care sub-factor may include measurement information from patient satisfaction surveys. The utilization sub-factor may include information related to frequency of selected procedures, such as well-child visits, inpatient utilization, and/or identification of alcohol and other drug services. The resource use sub-factor may include information regarding resource use for patients with conditions, such as, but not limited to, diabetes, cardiovascular conditions, hypertension, chronic obstructive pulmonary disease, and/or asthma. The health plan information sub-factor may include information on board certifications and/or membership diversity for a patient's health plan.

Referring now to FIG. 4, as described above with respect to FIG. 2, the list of provider candidates may be generated at block 400 through use of or with the assistance of an AI engine, such the AI engine 245 of FIG. 2.

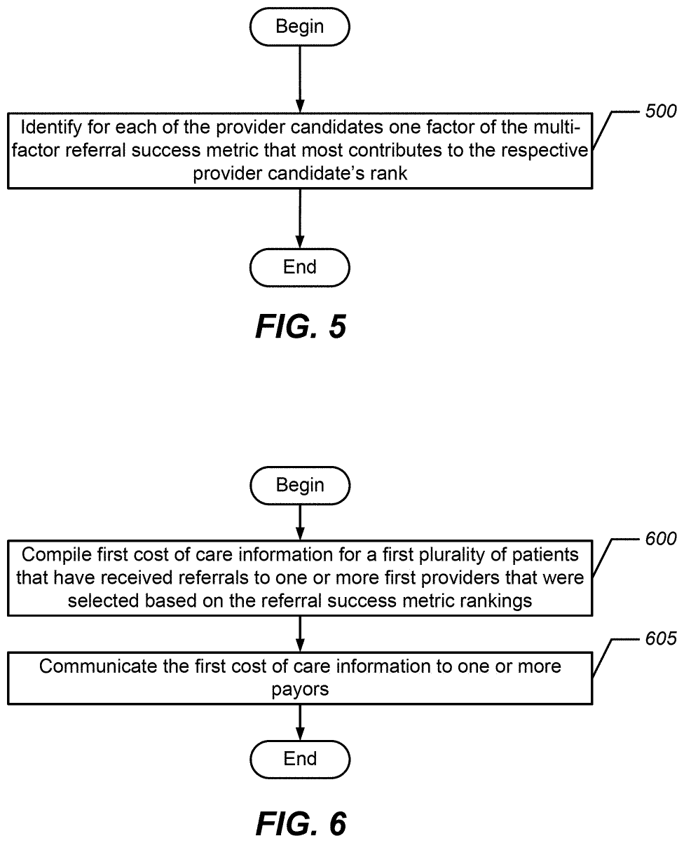

Referring now to FIG. 5, the provider candidates may be ranked and include an identification of the one factor of the multi-factor success metric that most contributes to the provider candidate's rank at block 500. This may assist the user, such as a primary care provider, in making a final selection from the list of ranked provider candidates, particularly when several candidates have similar rankings. In some embodiments, the ranked list of provider candidates may include the metric information for one or more of the multiple factors that comprise the multi-factor success metric, such as cost, complication, and quality, including one or more quality sub-factors, for each of the candidates.

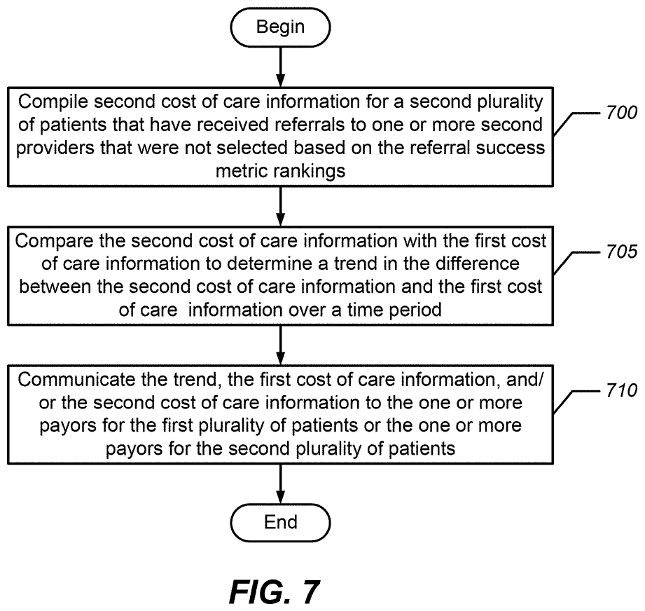

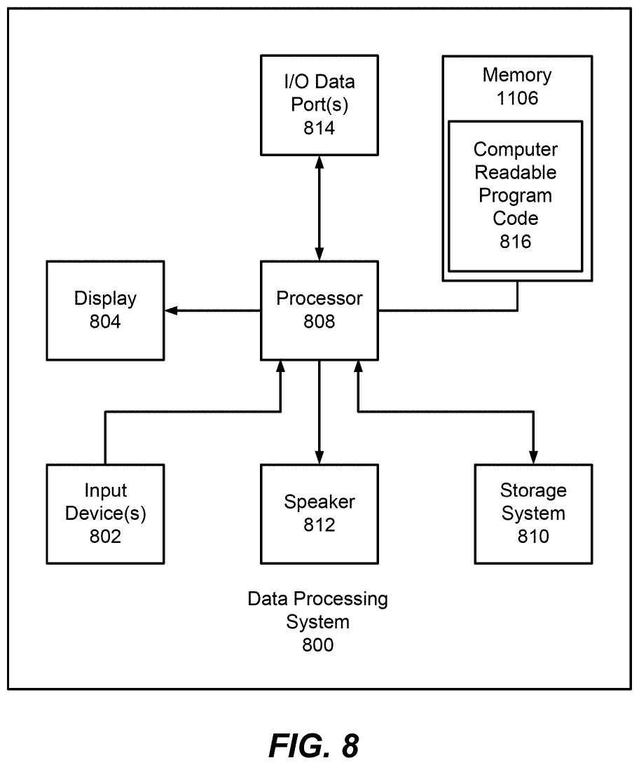

As described above, payors may seek information on which providers use the referral recommendation system to evaluate whether the cost of care is decreasing for those patients who receive provider referrals that were identified through the provider referral recommendation system relative to patients who receive referrals without using the provider referral recommendation system. Thus, referring to FIG. 6, first cost of care information may be compiled for patients that have received referrals that were selected from a ranked list generated by the provider referral recommendation system based on a multi-factor referral success metric at block 600 and this first cost of care information may be communicated to one or more payors at block 605. Referring now to FIG. 7, second cost of care information may be compiled for patients that have received referrals that were selected without using the provider referral recommendation system based on a multi-factor success metric at block 700. A trend may be determined based on the difference between the first cost of care of patients that received provider referrals using the provider referral recommendation system and those patients that received provider referrals without using the provider referral recommendation system over time at block 705. The trend, and the first cost of care information for the patients that received provider referrals through the provider referral recommendation system, and/or the second cost of care information for the patients that received provider referrals without using the provider referral recommendation system may be communicated to one or more payors for the patients that received provider referrals using the provider referral recommendation system and/or for one or more payors for patients that received provider referrals without using the provider referral recommendation system. Payors may use this information to evaluate the effectiveness of the provider referral recommendation system and make decisions on whether to encourage primary care providers, for example, to use the provider referral recommendation system in their practices.

Referring now to FIG. 8, a data processing system 800 that may be used to implement the referral server 140 of FIG. 1, in accordance with some embodiments of the inventive concept, comprises input device(s) 802, such as a keyboard or keypad, a display 804, and a memory 806 that communicate with a processor 808. The data processing system 800 may further include a storage system 810, a speaker 812, and an input/output (I/O) data port(s) 814 that also communicate with the processor 808. The processor 808 may be, for example, a commercially available or custom microprocessor. The storage system 1110 may include removable and/or fixed media, such as floppy disks, ZIP drives, hard disks, or the like, as well as virtual storage, such as a RAMDISK. The I/O data port(s) 814 may be used to transfer information between the data processing system 800 and another computer system or a network (e.g., the Internet). These components may be conventional components, such as those used in many conventional computing devices, and their functionality, with respect to conventional operations, is generally known to those skilled in the art. The memory 806 may be configured with computer readable program code 816 to facilitate generation of a ranked list of candidate providers for performing a procedure or treatment based on a multi-factor referral success metric according to some embodiments of the inventive concept.

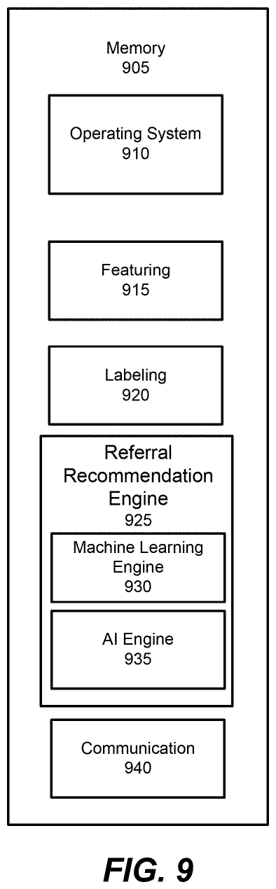

FIG. 9 illustrates a memory 905 that may be used in embodiments of data processing systems, such as the referral server 140 of FIG. 1 and the data processing system 800 of FIG. 8, respectively, to facilitate provider referral recommendation based on a multi-factor referral success metric according to some embodiments of the inventive concept. The memory 905 is representative of the one or more memory devices containing the software and data used for facilitating operations of the referral server 140 and referral recommendation engine 145 as described herein. The memory 905 may include, but is not limited to, the following types of devices: cache, ROM, PROM, EPROM, EEPROM, flash, SRAM, and DRAM. As shown in FIG. 9, the memory 905 may contain five or more categories of software and/or data in an AI embodiment: an operating system 910, a featuring module 915, a labeling module 920, a referral recommendation engine module 925, and a communication module 940. In particular, the operating system 910 may manage the data processing system's software and/or hardware resources and may coordinate execution of programs by the processor. The featuring module 915 may be configured to perform one or more of the operations described above with respect to the featuring modules 225, 265 and the flowcharts of FIGS. 3-7. The labeling module 920 may be configured to perform one or more of the operations described above with respect to the labeling module 230 and the flowcharts of FIGS. 3-7. The referral recommendation engine 925 may comprise a machine learning engine module 930 and an AI engine module 935. The machine learning engine module 930 may be configured to perform one or more operations described above with respect to the machine learning engine 240 and the flowcharts of FIGS. 3-7. The AI engine module 935 may be configured to perform one or more operations described above with respect to the AI engine 245 and the flowcharts of FIGS. 3-7. In a non-AI implementation, the referral recommendation engine module 925 may be embodied using procedural and/or objected oriented computer readable program code, for example, and may be configured to carry out one or more of the operations described above with respect to FIGS. 3-7. The communication module 940 may be configured to support communication between, for example, the referral server 140 and the health care facility/payor interface server 130 and/or providers 110a, 110b, and 110c.

Although FIGS. 8-9 illustrate hardware/software architectures that may be used in data processing systems, such as the referral server 140 of FIG. 1 and the data processing system 800 of FIG. 8, respectively, in accordance with some embodiments of the inventive concept, it will be understood that embodiments of the present invention are not limited to such a configuration but is intended to encompass any configuration capable of carrying out operations described herein.

Computer program code for carrying out operations of data processing systems discussed above with respect to FIGS. 1-9 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the referral server 140 of FIG. 1 and the data processing system 800 of FIG. 8 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-9 may be used to facilitate generation of a ranked list of provider candidates for a procedure or a treatment that is based on a multi-factor success metric according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 905 when coupled to a processor includes computer readable program code that, when executed by the processor, causes the processor to perform operations including one or more of the operations described herein with respect to FIGS. 1-9.

Some embodiments of the inventive concept described herein may provide a provider referral recommendation system that is based on a multi-factor success metric and may provide a geographically appropriate list of provider candidates for a particular procedure or treatment for a patient. The provider candidates may be evaluated and ranked for a particular procedure based on multiple factors, such as cost, complications, and quality. This ranked list may be provided to a primary care provider for use in selecting a provider as a referral for further patient care. By evaluating the providers based on these metrics, the total cost of care for the patient may be reduced through use of, for example, specialists that exhibit higher quality, lower, cost, and few complications in caring for patients.

Further Definitions and Embodiments:

In the above description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random-access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving first information associated with a patient, the first information including a procedure or treatment that is recommended for the patient; and
generating a list of provider candidates for referring to the patient based on the first information associated with the patient, the list of provider candidates being ranked based on a multi-factor referral success metric;
wherein the multi-factor referral success metric comprises a cost factor, a complication factor, and a quality factor.

2. The method of claim 1, wherein the first information further includes geographic information associated with the patient.

3. The method of claim 1, wherein the complication factor comprises:
unanticipated problems that have arisen following, and are a result of, one or more previous procedures, treatments, or patient illnesses.

4. The method of claim 1, wherein the cost factor comprises:
expenses associated with past performance of the procedure or the treatment that is recommended for the patient.

5. The method of claim 1, wherein the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

6. The method of claim 1, wherein generating the list of provider candidates comprises generating the list of provider candidates using an artificial intelligence engine.

7. The method of claim 1, further comprising:
identifying, for each of the provider candidates, one factor of the multi-factor referral success metric that most contributes to the respective provider candidate's rank in the list of provider candidates.

8. The method of claim 1, further comprising:
compiling first cost of care information for a first plurality of patients that have received referrals to one or more first providers that were selected based on a plurality of lists of provider candidates generated therefor and ranked based on the multi-factor referral success metric, respectively; and
communicating the first cost of care information to one or more payors for the first plurality of patients.

9. The method of claim 8, further comprising:
compiling second cost of care information for a second plurality of patients that have received referrals to one or more second providers that were not selected based on a plurality of lists of provider candidates generated therefor and ranked based on the multi-factor referral success metric, respectively;
comparing the second cost of care information with the first cost of care information to determine a trend in the difference between the second cost of care information and the first cost of care information over a time period; and
communicating the trend to the one or more payors for the first plurality of patients or one or more payors for the second plurality of patients.

10. A system, comprising:
a processor; and
a memory coupled to the processor and comprising computer readable program code embodied in the memory that is executable by the processor to perform operations comprising:
receiving first information associated with a patient, the first information including a procedure or treatment that is recommended for the patient; and
generating a list of provider candidates for referring to the patient based on the first information associated with the patient, the list of provider candidates being ranked based on a multi-factor referral success metric;
wherein the multi-factor referral success metric comprises a cost factor, a complication factor, and a quality factor.

11. The system of claim 10, wherein the first information further includes geographic information associated with the patient;
wherein the complication factor comprises:
unanticipated problems that have arisen following, and are a result of, one or more previous procedures, treatments, or patient illnesses;
wherein the cost factor comprises:
expenses associated with past performance of the procedure or the treatment that is recommended for the patient; and
wherein the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

12. The system of claim 10, wherein generating the list of provider candidates comprises generating the list of provider candidates using an artificial intelligence engine.

13. The system of claim 10, wherein the operations further comprise:
identifying, for each of the provider candidates, one factor of the multi-factor referral success metric that most contributes to the respective provider candidate's rank in the list of provider candidates.

14. The system of claim 10, wherein the operations further comprise:
compiling first cost of care information for a first plurality of patients that have received referrals to one or more first providers that were selected based on a plurality of lists of provider candidates generated therefor and ranked based on the multi-factor referral success metric, respectively; and
communicating the first cost of care information to one or more payors for the first plurality of patients.

15. The system of claim 14, wherein the operations further comprise:
compiling second cost of care information for a second plurality of patients that have received referrals to one or more second providers that were not selected based on a plurality of lists of provider candidates generated therefor and ranked based on the multi-factor referral success metric, respectively;
comparing the second cost of care information with the first cost of care information to determine a trend in the difference between the second cost of care information and the first cost of care information over a time period; and
communicating the trend to the one or more payors for the first plurality of patients or one or more payors for the second plurality of patients.

16. A computer program product, comprising:
a non-transitory computer readable storage medium comprising computer readable program code embodied in the medium that is executable by a processor to perform operations comprising:
receiving first information associated with a patient, the first information including a procedure or treatment that is recommended for the patient; and
generating a list of provider candidates for referring to the patient based on the first information associated with the patient, the list of provider candidates being ranked based on a multi-factor referral success metric;
wherein the multi-factor referral success metric comprises a cost factor, a complication factor, and a quality factor.

17. The computer program product of claim 16, wherein the first information further includes geographic information associated with the patient;
wherein the complication factor comprises:
unanticipated problems that have arisen following, and are a result of, one or more previous procedures, treatments, or patient illnesses;
wherein the cost factor comprises:
expenses associated with past performance of the procedure or the treatment that is recommended for the patient; and
wherein the quality factor comprises a plurality of quality sub-factors, the plurality of quality sub-factors comprising an effectiveness of care sub-factor, an availability of care sub-factor, an experience of care sub-factor, a utilization sub-factor, a resource use sub-factor, and a health plan information sub-factor.

18. The computer program product of claim 16, wherein generating the list of provider candidates comprises generating the list of provider candidates using an artificial intelligence engine.

19. The computer program product of claim 16, wherein the operations further comprise:
identifying, for each of the provider candidates, one factor of the multi-factor referral success metric that most contributes to the respective provider candidate's rank in the list of provider candidates.

20. The computer program product of claim 16, wherein the operations further comprise:
compiling first cost of care information for a first plurality of patients that have received referrals to one or more first providers that were selected based on a plurality of lists of provider candidates generated therefor and ranked based on the multi-factor referral success metric, respectively;
compiling second cost of care information for a second plurality of patients that have received referrals to one or more second providers that were not selected based on a plurality of lists of provider candidates generated therefor and ranked based on the multi-factor referral success metric, respectively;
comparing the second cost of care information with the first cost of care information to determine a trend in the difference between the second cost of care information and the first cost of care information over a time period; and
communicating the first cost of care information, the second cost of care information, and the trend to one or more payors for the first plurality of patients or one or more payors for the second plurality of patients.
